(19) United States
(12) Patent Application Publication
(10) Pub. No.: US 2012/0165853 A1
(43) Pub. Date: Jun. 28, 2012

(54) Femoral Vein Catheter for Improving Cardiac Output, Drug Delivery and Automated CPR Optimization

(75) Inventors: Igor Wilhelmus Franciscus Paulussen, Nuenen (NL); Pierre Hermanus Woerlee, Valkenswaard (NL); Gerrit Jan Noordergraaf, Nuenen (NL); Abraham Noordergraaf, Haverford, CT (US)

(73) Assignee: KONINKLIJKE PHILIPS ELECTRONICS N.V., Eindhoven (NL)

(21) Appl. No.: 13/392,602

(22) PCT Filed: Sep. 9, 2010
(86) PCT No.: PCT/IB2010/054063

§ 371 Date: Feb. 27, 2012

(30) Foreign Application Priority Data
Sep. 18, 2009 (EP) 09170644.0

(51) Int. Cl. A61M 29/00

(52) U.S. Cl. 606/191

(57) ABSTRACT

A blood flow control device, comprising a flow influencing element arranged to be placed in the vena cava of a human during cardiopulmonary resuscitation and controllable between a non-to-low-flow state in which the flow influencing element substantially reduces a blood flow within the vena cava, and a flow state, in which the flow influencing element allows substantially unreduced blood flow, responsive to an existing or a predicted pressure difference between an upstream area and a downstream area of the flow influencing element. The blood flow control device is capable of reducing retrograde blood flow during the compression phase of CPR and thus improves the efficiency of CPR and blood perfusion. The blood flow control device can also be used for the administration of drugs almost directly to the heart, as well as for measuring physiological and chemical properties, such as blood gases.

### FIELD OF THE INVENTION

The field of the present invention relates to a blood flow control device, for example for use during cardiopulmonary resuscitation (CPR) of a patient.

### DESCRIPTION OF THE RELATED ART

Sudden Cardiac Arrest (SCA) remains one of the main causes of death in the western world. The resulting whole body ischemia after the SCA disturbs a wide range of cell processes, leading to severe cell damage and death unless acute medical care is available. It has been reported that the probability for survival after sudden cardiac arrest decreases linearly with 7-10% per minute of arrest time. Starting within about 4 minutes, a minimum amount of perfusion (induced by CPR or other means) is required to support cells and organs until further treatment (e.g. defibrillation) can be applied.

Perfusion by CPR is at a very low level even if carried out perfectly, with estimates of a maximum of 30% of the original cardiac output. In addition, forward, as well as backward blood flow may be generated by CPR as well as generalized, intravascular, volume trapping. Ischemia and cell damage during CPR may be aggravated by this phenomenon.

A CPR-induced flow abnormality is the so-called sloshing phenomenon. In the medical literature, several explanations for the occurrence of sloshing have been and are being discussed. One theory suggests that the cardiac chambers within the pericardium are simultaneously compressed, forcing blood into the lower pressure in- and outflow vascular tracts, the motion completely following the pressure gradients. Another suggestion is that the generalized intrathoracic pressure increase induced by chest compressions may cause gradients in vascular and non-vascular tissues alike all throughout the thoracic cavity. These pressures will induce flows to other (local) lower-pressure area's or may induce only a local pressure peak without flow if both up-stream and down-stream vasculature are closed by the pressure. This introduces a specific factor in time sensitivity and effect of tissue response to the pressure wave. The compression of the atria and ventricles may or may not act simultaneously to the compressions of the heart. It is also very likely that the central veins collapse, since they are subjected to the force being disseminated within the thoracic cavity. The time sensitivity in the pressure effect on the intrathoracic vasculature as well as on the cardiac chambers may induce blood volume to move from high to low pressure areas or to protected (e.g. capacitance) vessels. Focusing on the inflow tracts at the right atrium, blood can move both forward (i.e. into/through the heart) and backward (i.e. retrograde into the vena cava or even out of the thoracic cavity), The blood volume present in the central veins and the right atrium therefore may just move back and forth instead of moving forward while an intravascular pressure curve suggests otherwise. When sloshing occurs, the net, forward, blood flow may be low or even absent.

Another aspect in resuscitation is the administration of drugs (vasopressor, anti arrhythmic, etc). These drugs need to be supplied to their effector sites, in or via the central circulation. Distribution may be influenced by the location of (peripheral) injection site, and/or poor perfusion during manual CPR, as well as short degradation times for the medication. Assuring central availability, to ensure distribution through effective forward flow, may be an aspect which might allow for further optimization of the CPR.

### BRIEF SUMMARY OF THE INVENTION

It would be desirable to reduce or even avoid retrograde flow in the (inferior) vena cava during the compression phase of CPR. Moreover, it would be desirable to reduce the sloshing phenomenon and the trapping of blood. It would also be desirable that any device intended for this purpose be easy and safe to insert. In order to address at least one of these concerns and/or other concerns, a blood flow control device is proposed. The blood flow control device comprises a flow influencing element arranged to be placed in the vena cava of a human during cardiopulmonary resuscitation. The flow influencing element is controllable between a non-to-low-flow state in which the flow influencing element substantially reduces a blood flow within the vena cava, and a flow state, in which the flow influencing element allows substantially unreduced blood flow, responsive to an existing or a predicted pressure difference between an upstream area and a downstream area of the flow influencing element.

Usually, there are no natural valves between the right atrium and the inferior vena cava. The same is true for the abdominal pool. This is usually not a problem with a heart functioning in the normal manner, because respiration and the cardiac cycle creates a pressure gradient which causes blood in the inferior vena cave to flow into the right atrium. However, when the heart is driven by external compressions the normally finely tuned sequence of the compressions of the atria and ventricles is no longer assured. The flow influencing element of the proposed blood flow control device may be regarded as assuming the role of a venous valve.

In the non-to-low-flow state, which may also be called reduced flow state, the flow influencing element substantially limits or even blocks the blood flow within the vena cava. In contrast, in the flow state the flow influencing element should present only a small flow resistance to the blood flow. When thoracic compressions are applied to the patient during CPR, pressure in the right atrium fluctuates. With respect to the flow influencing element, the right atrium is usually in the downstream area of the flow influencing element. The other side of the flow influencing element, e.g. the abdominal region, shall be considered to be the upstream area of the flow influencing element. Pressure fluctuations in the right atrium (or at another place) that are caused by the CPR compressions may be used to synchronize the state-toggling operation of the flow influencing element. This can be achieved by measuring an existing pressure difference between the upstream area and the downstream, or by forecasting a predicted pressure difference. It may indeed be possible to predict the pressure difference based on previous measurements, or by evaluating a trend in the temporal evolution of the pressure difference. If the pressure difference can be sufficiently reliably predicted, then it may be possible to anticipate the state-toggling action of the flow influencing element, which may in turn improve the efficiency of the blood flow control device.

It would also be desirable that the blood flow control device has good sensitivity regarding the detection of compressions and offers some degree of adjustability. These concerns and/or possibly other concerns are addressed by the blood flow control device further comprising a compression sensor and a control unit. The compression sensor is arranged to detect compressions related to an aspect of cardiopulmonary resuscitation. The control unit is connected to the compression sensor and to the flow influencing element. By means of the compression sensor the control unit detects an intrathoracic pressure change or movement of the thoracic wall or compression, and sends a signal to the flow influencing element causing the flow influencing element to assume the non-to-low-flow state. The compression sensor facilitates reliable detection of compressions that are performed during CPR. The control unit receives measurements from the compression sensor and processes them in order to derive a drive signal for the flow influencing element. The control unit may have one or more adjustable parameters, such as thresholds or delays. It is possible that by adjusting some parameters of the control unit a more optimized operation of the blood flow control device can be achieved.

It would be desirable that the compression sensor could measure a physical quantity that is related to the compressions. In an embodiment this concern is addressed by the compression sensor being arranged to measure compression force and/or chest displacement and/or intrathoracic pressure change and/or intravascular flow. The proposed physical quantities have a mechanical or fluid dynamical relation to the administration of compressions.

It would be further desirable that the blood flow control device is easy and safe to insert, as it is likely to be employed during an emergency situation. In an embodiment this concern and/or possible other concerns are addressed by the blood flow control device further comprising a catheter and wherein the compression sensor is placed in a tip of the catheter. The catheter may for example be inserted via a femoral vein (femoral cannulation). Placing the compression sensor in the tip of the catheter brings the compression sensor to a place where the effects of the administration of chest compressions are usually detectable when the compression sensor is placed in the tip of the catheter. The compression sensor is integrated in the tip of the catheter in the vicinity of the flow influencing element. Only one insertion procedure needs to be performed for positioning the flow influencing element and the compression sensor at the intended site. Thus, the blood flow control device is quickly ready for operation. The tip of the catheter may be spaced from the flow influencing element so that the compression sensor is placed closer to the heart or even within the right atrium.

Depending on the situation and user preferences it may also be desirable to place the compression sensor independently from the rest of the blood flow device. In an embodiment it is proposed that the compression sensor is arranged to be positioned at the outside of the body of the patient. For example, a pressure-sensitive pad may be positioned on the sternum of the patient so that the time sensitive compression force/displacement curve can be directly measured at the interface between the palm of a rescuer and the chest of the patient or victim. It is also possible that the blood flow control device comprises several compression sensors, for example an internal compression sensor and an external compression sensor. The control unit of the blood flow control device could then analyze the measurements of both the internal and the external compression sensors.

In an embodiment it is proposed that the compression sensor is arranged to be positioned within the thoracic cavity. It would also be desirable to have the ability to measure key physiological parameters, which would enable poor quality chest compression (e.g. force displacement) to be recognized and corrected by this feedback modality. In an embodiment this concern is addressed by the blood flow control device further comprising at least one of a physiological sensor and a chemical sensor. The physiological sensor is arranged to measure vital physiological parameters. The chemical sensor is arranged to measure bio-chemistry parameters. Measurement of parameters related to perfusion such as blood gases (PvO₂, PvCO₂), pH, blood pressure, blood flow, etc. in relation to the CPR activities would be desirable. The physiological sensor and/or the chemical sensor may be positioned in the tip of the catheter, within the thoracic cavity or outside of the body of the human, depending on the parameters values sought.

It would be further desirable that the blood flow control device can be positioned in the vena cava in an efficient manner. In an embodiment this concern is addressed by the flow influencing element comprising an inflatable element or cusp shaped device. An inflatable element or a cusp shaped device provides good adaptability to the interior form of the vessel. Thus, leakage between the wall of the vessel and the flow influencing element can be substantially prevented or reduced while trauma to the vessel wall is limited or avoided. The (deflated) inflatable element and the cusp shape element are also relatively easy to advance from their insertion point to their final position just outside the right atrium. To this end, the inflatable element is deflated during the transport from the insertion site to the section of the vessel were the flow influencing element is intended to be positioned. The cusp shape device may be flexible enough to adapt its form to the veins that it traverses during the transport.

In an embodiment it is proposed that the blood flow control device further comprises a pressure source and a pipe for connecting the pressure source with the inflatable element or the cusp shape device for deflating and/or inflating the inflatable element or for manually adjusting the form of the cusp shape device. By using a pressure source deflating and inflating can be performed in a semi-automatic or in an automatic manner. This is useful when deflating and inflating is also used for toggling the state of the flow influencing element between the non-to-low-flow state and the flow state. The pressure source may be a pump or a high pressure reservoir. The pressure source may be connected to the pipe by one or several control valves.

It would be also desirable that the flow state toggling action of the flow influencing element can be performed sufficiently fast so that it can be in synchronicity with the administration of the chest compressions (or more specifically the intrathoracic pressure changes operating on the vena cava and the right heart). In an embodiment this concern is addressed by the flow influencing element comprising a functional valve. Depending on the design of the functional valve its response can be sufficiently fast so that the valve can be opened and closed once per compression cycle. For example, the valve could be of the butterfly design or the flap design. Furthermore, the positioning of the flow influencing element is not or only marginally influenced by the toggling action, if the flow influencing element comprises a valve. In other words, the positioning function is, in this case, substantially separate from the flow state control function.

It would further be desirable in some situations to be able to actively control the flow state toggling. In an embodiment this concern is addressed by the blood flow control device further comprising a catheter having a first lumen for the transmission of a drive signal to the flow influencing element for controlling the flow influencing element between the non-to-low flow state and the flow state. The first lumen may contain the pipe for connecting the pressure source with the inflatable element, or the first lumen and the pipe may coincide.

It would further be desirable that during CPR only one (minimally) invasive intervention is needed. This also applies to the need to administer drugs prior to or during the cardiopulmonary resuscitation. Another concern relative to drug administration during CPR is that blood perfusion is usually relatively low during a sudden cardiac arrest. Therefore it can be assumed that it would be helpful and more efficient to deliver the drugs directly to that part of the body where they are needed. In an embodiment this concern and/or other concerns are addressed by the catheter further comprising at least a second lumen arranged to be used for the delivery of substances to a location in the vicinity of the flow influencing element. The flow influencing element is usually positioned close to the heart. This is the part of the body where at least some blood perfusion can be expected during CPR. Furthermore, drugs administered during CPR are usually intended to stimulate the heart, as well as pass through the heart to the peripheral effector sites (e.g. the arterioles) so that a faster and more efficient reaction to the drugs can be expected, if the drugs are delivered close to the heart or directly to the heart.

Based on its intended usage (for example during an emergency in the field, as opposed to a usage in a hospital environment) and user preferences it may be desirable that the blood flow control device avoids complexity and yet offers satisfactory user and technical control over its performance. In an embodiment this concern is addressed by the flow influencing element functioning in the manner of a check valve. A check valve is controlled by the pressures at its upstream side and its downstream side in a substantially self-regulatory manner. With the flow influencing element functioning in the manner of a check valve it is not necessary to have a great deal of additional equipment outside of the body. Optimally, no active elements are needed for the operation of a check valve which would require some kind of energy source, such as a battery, if the gradient is sufficient for this purpose.

It would further be desirable to combine equipment for automated cardiopulmonary resuscitation with a blood flow control device as described above. In an embodiment, this concern is addressed by the blood flow control device further comprising a control signal interface for receiving a control signal from an automated cardiopulmonary resuscitation apparatus. The control signal causes the flow influencing element to toggle between the non-to-low-flow state and the flow state in a synchronized manner with the automated cardiopulmonary resuscitation. An automated cardiopulmonary resuscitation apparatus is often used nowadays for long-term life support. For long-term life support it is desirable that blood perfusion is maintained at a sufficient level to support vital organ perfusion. The reason for this is that organs that are poorly supplied with blood may be severely damaged, especially the brain. The blood flow control device according to the teachings disclosed herein is capable of improving the blood perfusion performance. In the case of an automated CPR the compression frequency is usually regular and within a limited range with respect to frequency and controlled very accurately so that the flow state toggling action of the flow influencing element can be time synchronized. In this way, a phase shift can be applied to the toggling action which could, for example, correct for the transition time between the non-to-low-flow state and the flow state. This makes it possible to have the non-to-low-flow state begin just before the compression phase.

In an embodiment it is proposed that the flow influencing element is arranged to be introduced into the vena cava by means of a percutaneous procedure, e.g. a femoral cannulation procedure. Femoral cannulation is a (minimally) invasive approach that is assumed to be well suited for the purpose of inserting a blood flow control device in the vena cava. As options to this an open technique (e.g. cut down procedure) may be envisioned. This technique is well suited for controlled and less controlled environments, can be performed without interrupting (automated) cardiopulmonary resuscitation, and has a limited spectrum of intrinsic risks.

It would also be desirable that the blood flow control device reacts to a situation when natural circulation returns or to moments or periods of time during which chest compressions are not being administered. In an embodiment this concern is addressed by the flow influencing element remaining in the flow state when return of spontaneous circulation (ROSC) is achieved or when chest compressions are paused or stopped. This may be achieved by defining a resting state or quiescent state for the flow influencing element, for example by controlling the actuator in a corresponding manner or by elastically soliciting the flow influencing elements to the flow state position or shape. When natural blood perfusion returns, the flow influencing element may not interfere with the blood flow, in particular under natural but low flow conditions when no chest compressions are administered anymore which may control the toggling action of the flow influencing element. Having a well defined resting position or resting shape of the flow influencing element might prevent that the blood flow control device has adverse effects on the natural blood perfusion.

It is possible that several or all of the features described above are implemented in a blood flow device. Such a blood flow control device might improve blood perfusion by reducing retrograde blood flow, it may facilitate the administration of drugs, and/or it may comprise sensors for a measurement of the quality and personalization of CPR.

The teachings disclosed herein may also be used in the context of a method for blood flow control. A method for blood flow control might contain the following actions:
placing a flow influencing element in the vena cava of a human during cardiopulmonary resuscitation,
   wherein the flow influencing element is controllable between a non-to-low-flow state and a flow state, responsive to an existing or a predicted pressure difference between an upstream area and a downstream area of the flow influencing element.

The method may further comprise actions that correspond to the features described in the description and/or in the claims directed at the blood flow control device.

The teachings disclosed herein may also be used in the context of a computer program product comprising instructions for a processor for controlling a blood flow control device. The computer program product may further comprise instructions that correspond to the features described in the description and/or in the claims directed at the blood flow control device.

These and other aspects of the invention will be apparent from and illustrated with reference to the embodiment(s) described herein after.

### BRIEF DESCRIPTION OF THE DRAWINGS

FIG. 1 shows an overview of the placement of a blood flow control device.

FIG. 2 shows an embodiment of a flow influencing element in the non-to-low-flow state (left) and in the flow state (right).

FIG. 3 shows another embodiment of a flow influencing element.

FIGS. 4 and 5 respectively show a front view and a sectional view of a further embodiment of a flow influencing element.

FIG. 6 shows a sectional view of yet another embodiment of a flow influencing element.

FIG. 7 shows a time diagram of several blood flow measurements taken at a healthy person.

FIG. 8 shows a time diagram of several blood flow measurements taken during the administration of CPR.

FIG. 9 shows two time diagrams illustrating a relationship between compression force and pressure within an inflatable element as performed by some embodiments of a blood flow control device.

FIG. 10 shows a sectional view of a further embodiment of a flow influencing element.

FIG. 11 shows a schematic block diagram of the various sub-units of the blood flow control device.

### DETAILED DESCRIPTION OF THE EMBODIMENTS

The invention will now be described on the basis of the drawings. It will be understood that the embodiments and aspects of the invention described herein are only examples and do not limit the protective scope of the claims in any way. The invention is defined by the claims and their equivalents. It will also be understood that features of one aspect can be combined with a feature of a different aspect or aspects.

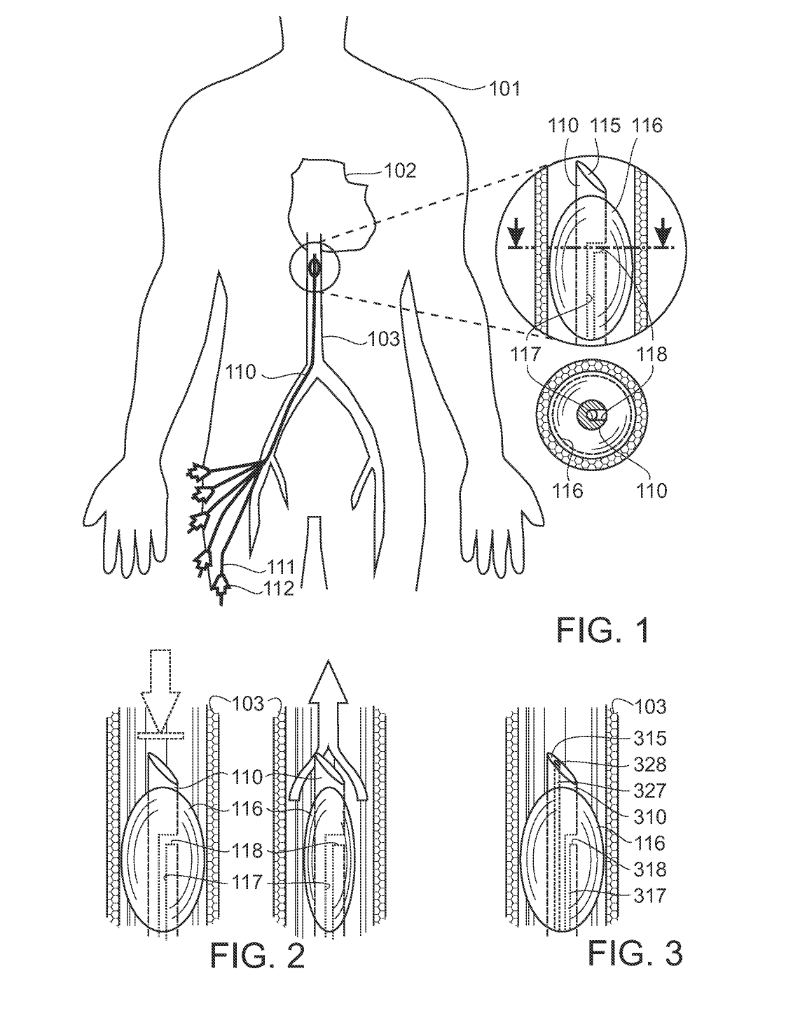

FIG. 1 shows in a schematic manner a human torso 101. Also illustrated are the heart 102, the inferior vena cava 103 and the right femoral vein 104. Prior to or during a CPR intervention a catheter-like device 110 is inserted via the right femoral vein 104 and the vena cava 103. The tip of the catheter-like device 110 comes to rest near the heart 102, provided the insertion of the catheter-like device 110 has been successful.

The right part of FIG. 1 shows a detailed view of the vena cava 103 and a flow influencing element at the tip of the catheter-like device 110. The blood flow control device may be regarded as a balloon-on-a-catheter placed into a large vein via a percutaneous route. The catheter-like device 110 has a rounded or slanted tip 115 that may be useful during the insertion procedure. The tip can be positioned so as to lie just caudally of the entrance to the right atrium. Slightly beneath the slanted tip 115 is an inflatable element 116, such as a balloon. The main effect of the inflation of the balloon during the compression phase of CPR is to block the vena cava avoiding retrograde blood flow. Deflation during CPR diastolic permits substantially unhindered venous return. The inferior vena cava might be blocked completely in this CPR systolic phase, thus avoiding retrograde blood flow towards the abdomen. The catheter-like device 110 also comprises at least one lumen 117 and an orifice 118, the function of which will be explained now in the context of an explanation of FIG. 2.

FIG. 2 shows the two states between which a flow influencing element may toggle during operation. The left picture of FIG. 2 shows the non-to-low-flow state of the flow influencing element. The inflatable element 116 is fully inflated so that it touches the wall of the vena cava 103. In doing so, any blood flow around the inflatable element is blocked and in particular a retrograde blood flow originating in the right atrium of the heart 102. In FIG. 2, as well in some of the other figures, a downstream area relative to the flow influencing element is situated above the illustrated flow influencing element. Likewise, an upstream area relative to the flow influencing element is situated beneath the illustrated flow influencing element. In the left picture of FIG. 2 the blocked retrograde blood flow is illustrated by a dashed arrow. The right picture of FIG. 2 shows the flow influencing element in the flow state. The inflatable element 116 is substantially deflated so that blood can flow around it. The alternating inflating and deflating action of the inflatable element 116 is controlled by means of the lumen 117 and the orifice 118. The lumen 117 is connected to a pressure source (not shown) outside the body of the patient 101. When the inflatable element is to be brought into the non-to-low-flow state (reduced flow state) then the pressure source urges a substance, such as air, water, etc., into the inflatable element, which is caused to expand (left picture of FIG. 2). In order to bring the flow influencing element into the flow state, the pressure source sucks a portion of the fluid out of the inflatable element by means of the orifice 118 and the lumen 117. Alternatively, the pressure force may just release or reduce the pressure so that the inflatable element returns to its contracted shape due to an elastic and/or resilient property. To summarize the operation of the embodiment shown in FIG. 2, the balloon is inflated during the compression phase of CPR, and is rapidly inflated at the onset of relaxation.

FIG. 3 shows another embodiment of the flow influencing element. In this embodiment the catheter-like device 310 comprises a first lumen 317, a first orifice 318, a second lumen 327 and a second orifice 328. The second lumen 321 and the second orifice 328, which is arranged in the tip 315, may be used for the delivery of drugs. The position of the second orifice 328 is such that drugs delivered though the second lumen 327 and the second orifice 328 are susceptible to be transported to the right atrium of the heart 102 during the next relaxation phase between two successive compressions. Drugs delivered in this manner usually reach the pulmonary circulation, the coronary circulation and the brain circulation quickly. Drugs that may be delivered by means of the second lumen 327 and the second orifice 328 are for example vaso-active drugs as well as other medication to the heart. It may be possible to provide more lumens so that an individual lumen or channel can be used for each drug, thus avoiding undesirable interactions between these drugs (e.g. epinephrine and sodium-bicarbonate). Drugs delivered in this manner are also often better distributed in the downstream vasculature due to effects such as reduced sloshing and better forward flow.

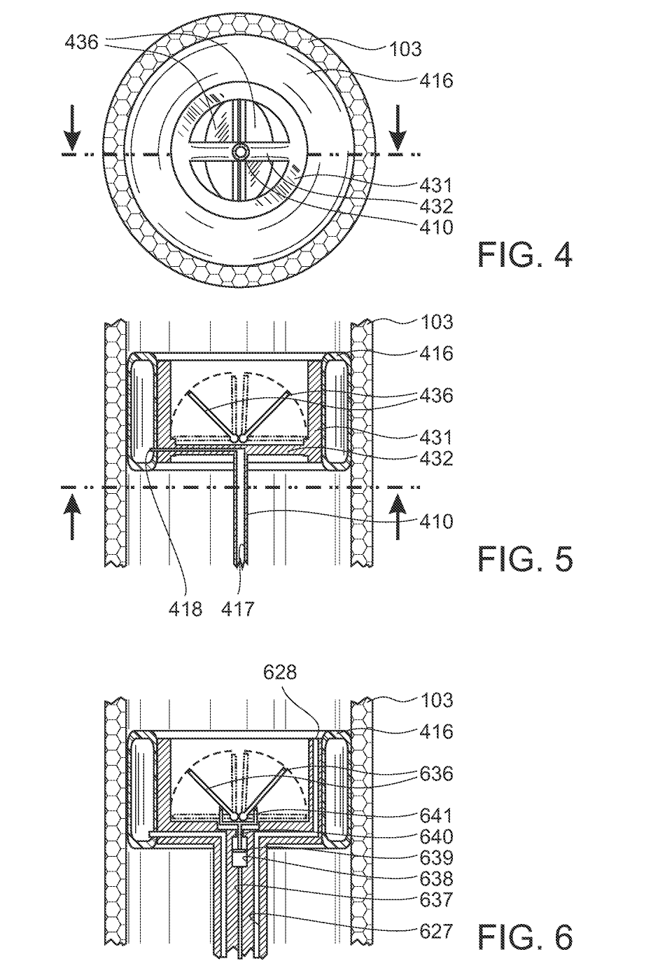

FIG. 4 shows a front view of another embodiment of the flow influencing device. FIG. 5 shows a corresponding sectional view. The flow influencing element comprises an inflatable element 416. However, contrary to the embodiment shown in FIGS. 1 to 3, the inflatable element 416 is not used to control the blood flow directly. The inflatable element 416 has a torus-like shape with a central opening. A frame or structure comprising a ring 431 and a strut 432 is disposed within the central opening. The frame 431, 432 may be of a relatively rigid material, such a stainless steel, a noble metal or plastic. The inflatable element 416 is usually made from an elastic material, such as rubber or silicon. Two flaps 436 are arranged within the ring 431 and rotatably attached thereto. The two flaps 436 form a butterfly-type valve. The strut 432 is connected to the catheter-like device 410 that is used to advance the flow influencing element within the vena cava to its operating position, and also to supply at least one control signal to the flow influencing element. For this reason, the catheter-like element 410 is hollow so that a fluid can act as a transmission medium for the control signal.

The function of the flow influencing element according to FIG. 4 becomes clear from the axial section shown in FIG. 5. A first lumen 417 within the catheter-like device 410 opens to the interior of the inflatable element 416 via the first orifice 418. With this arrangement it is possible to inflate and deflate the inflatable element 416. During the insertion procedure of the blood flow control device the inflatable element 416 is substantially deflated so that it has a smaller diameter than the diameter shown in FIGS. 4 and 5. FIGS. 4 and 5 do not necessarily show the proper dimensions that would allow an easy insertion procedure and a secure fixation at the intended position. The inflatable element could be dimensioned in a manner so that the ratio between the diameter in the inflated state and the deflated state is greater than illustrated in FIGS. 4 and 5. Once the flow influencing element is at the intended position, the inflatable element 416 is inflated via the first lumen 417 and the orifice 418. This causes the inflatable element to have tight contact with the wall of the vena cava 103. Once the inflatable element 416 has been inflated substantially no blood can flow around the flow influencing element anymore, that is between the wall of the vena cava and the inflatable element 416.

During CPR the two flaps 436 function in the manner of a check valve. When the pressure in the upstream area (beneath the flow influencing element in FIG. 5) is higher than the pressure in the downstream area (above the pressure influencing element in FIG. 5) then the flaps 436 will open and permit blood to flow through the flow influencing element. The open position of the flaps 436 corresponds to the flow state of the flow influencing element. When on the other hand the pressure at the downstream side of the flow influencing element is higher than on the upstream side, the two flaps 436 will close in an autonomous and/or self-regulating manner.

FIG. 6 shows another embodiment of a flow influencing element according to the teachings disclosed herein. The basic construction is similar to the embodiment shown in FIGS. 4 and 5. The embodiment shown in FIG. 6 differs from the previous embodiment in that the flow influencing element comprises two flaps 636 that are actively controllable from the outside of the body of the patient. Another difference is that a second lumen 627 is provided for drug delivery, in a similar manner to the embodiment shown in FIG. 3.

The mechanism that provides the active control of the flaps 636 comprises a third lumen 637, a cylinder 638, and a piston 639. The piston 639 is connected to a rod 640 which is in turn connected to a fork 641. The two ends of the fork 641 are connected to one of the flaps 636, respectively, by means of a pivot joint, an elastic joint, an abutment, etc. With this arrangement, a control signal for opening and closing the flaps 636 can be transmitted to the flow influencing element. A control signal consist of pressure variations in the third lumen 637 that cause the piston 639 to move up and down. The movement of the piston is transferred to the rod 640 and to the fork 641. This causes the flaps 636 to open or close in accordance with the control signal. The fluid within the lumen 637 may be pressurised air (i.e. an inert form such as CO2 or N2), water or another fluid that can be safely used within the blood circulation of a human body. Alternatively, it also possible to use a mechanical connection, such as a Bowden cable, or an electrical connection, in which case the cylinder-piston arrangement shown in FIG. 6 may be replaced by a solenoid.

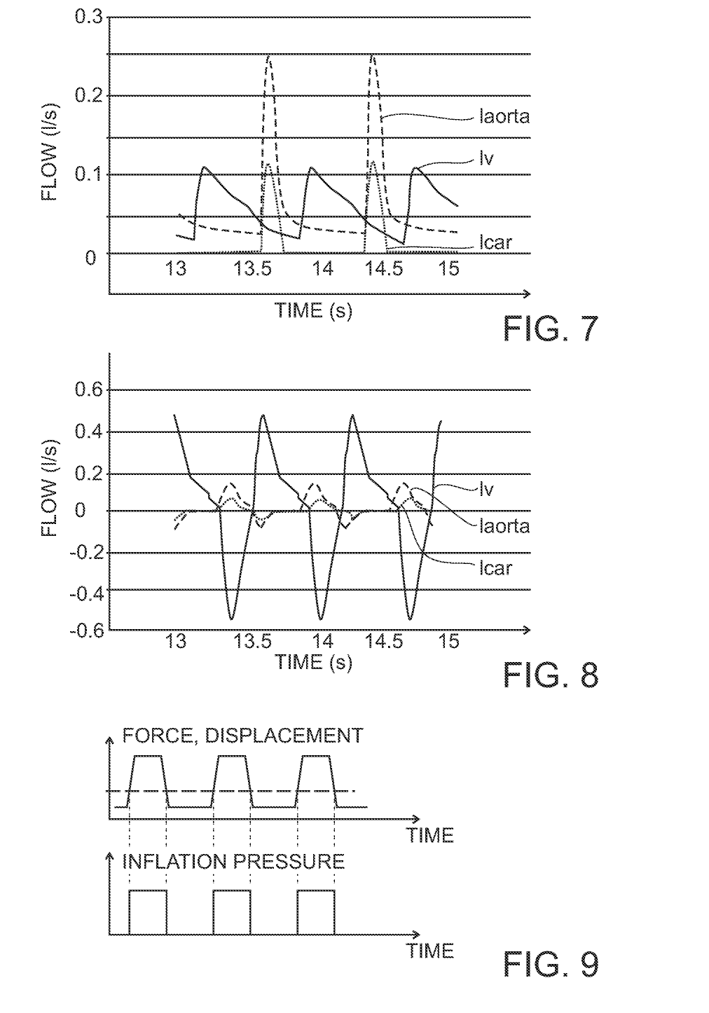

In FIG. 7 the exemplary flow in the aorta (laorta), the carotid artery (lcar) and the inferior vena cava (lv) are plotted for a normal beating heart. In FIG. 8, the same flows are plotted during CPR. As can be seen, very large sloshing flows are observed in the CPR case of FIG. 8. Especially the flow in the inferior vena cava lv shows that almost no net forward blood flow occurs, because the area under the negative parts of the plotted blood flow is almost equal to the area under the positive parts. To prevent sloshing and subsequent blood loss to the abdominal region, additional measures such as the ones described herein are helpful. Good results are expected if the flow in the inferior vena cava during the compression phase can be blocked as close to the distal inflow tract as possible.

FIG. 9 shows a combined time diagram of two signals that may be used by or within the blood flow control device according to the teachings disclosed herein. The upper part of FIG. 9 shows a measured signal of the force or the displacement that is related to the chest compressions performed by a rescuer or by an automated CPR. The dashed horizontal line represents a threshold at which a control unit of the blood flow control device assumes that a chest compression is currently being performed. When the force measurement or the displacement measurement exceeds the threshold (e.g. 10% of the minimum expected compression depth), the control unit may issue a control signal to the inflatable element of the embodiments shown in FIGS. 1 to 3, causing the inflatable element to expend. Thus, the flow influencing element is toggled into the non-to-low-flow state. Often, there is a small delay between the start of a compression and the expansion of the inflatable element. During this delay, the flow influencing element is not yet in the non-to-low-flow state so that a small amount of retrograde blood flow may occur. The same effect may occur towards the end of a compression.

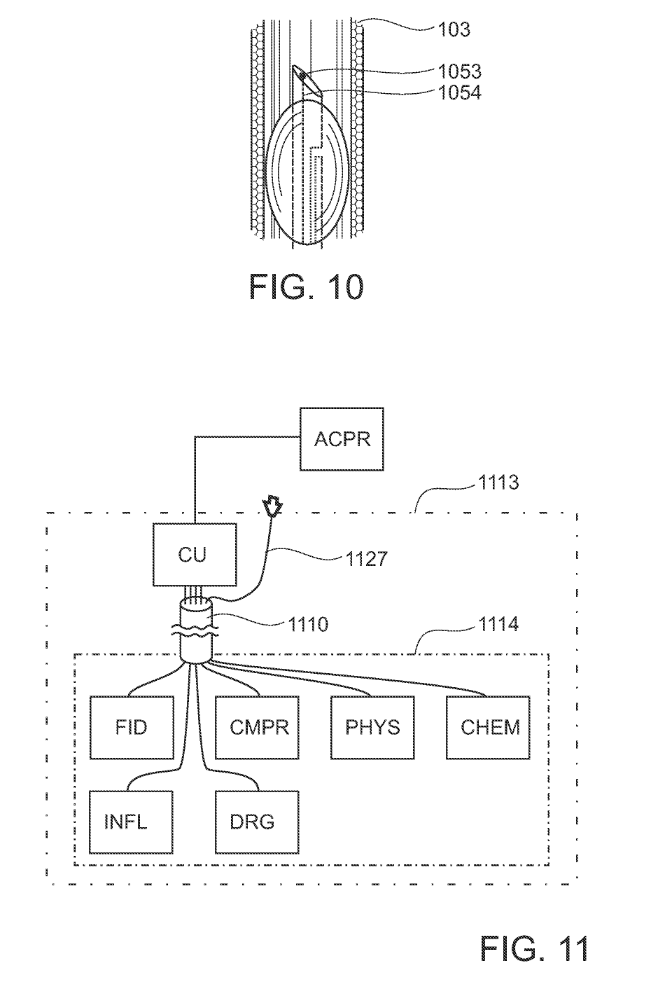

FIG. 10 shows an embodiment of the blood flow control device, and in particular the portion of the blood flow control device that is positioned in the inferior vena cava 103. The embodiment of FIG. 10 corresponds by and large to the embodiment shown in FIG. 2. Therefore, reference is made to FIG. 2 for those elements shown in FIG. 10 that have already been discussed in the context of FIG. 2. The embodiment shown in FIG. 10 additionally comprises a physiological or chemical sensor 1053. The sensor 1053 could also be a combination of several physiological and/or chemical sensors. A signal line 1054 connects the physiological or chemical sensor 1053 for example with a control unit of the blood flow control device. In FIG. 10, the physiological or chemical sensor 1053 is positioned at the tip of the catheter. Quantities of interest that may be measured by the physiological and/or chemical sensor are blood gases (PvO₂, PvCO₂), pH, blood pressure, blood flow, ions (K+, Na+, Ca₂+, Mg₂+, . . . ). These quantities can be used to optimize the quality of CPR as well as the quality of the resuscitation. Sensor data can also be used in a feed-back loop to optimize and personalize automatic CPR. Furthermore, part of the sensor data can be used for information concerning treatment of preventable causes of cardiac arrest (such as pH, ion balance, hypovolemia, . . . ).

FIG. 11 shows a schematic block diagram of the principal sub units (some of which are optional) of the blood flow control device according to the teachings disclosed herein. The blood flow control device 1113 typically comprises an external portion, and internal portion 1114 and a connection or link 1110 between the external portion and the internal portion 1114. The internal portion 1114 is intended to be inserted into the inferior vena cava 103, for example by means of a femoral cannulation. The basic component of the internal portion 114 is the flow influencing device FID. Various designs of the flow influencing device FID have been illustrated and discussed in FIGS. 2 to 6. The internal portion 1114 may further comprise various sensors, such as a compression sensor CMPR, a physiological sensor PHYS, and/or a chemical sensor CHEM. Another component of the internal portion 114 that may be present in some embodiments of the blood flow control device 1113 is an inflatable element INFL, such as the inflatable element 416 illustrated in FIGS. 4 to 6. In the context of FIGS. 4 to 6 the inflatable element 416 primarily served the purpose of fixing the internal portion 1114 at the intended position within the inferior vena cava 103. It is however possible to merge the flow influencing device FID and the inflatable element INFL, as illustrated in FIGS. 2 and 3. The internal portion 1114 may further comprise a drug delivery structure DRG, such as lumen 327, 627 and an orifice 328, 628, as shown in FIGS. 3 and 6.

The external portion may comprise a control unit CU, connectors for reading out the measurement signals of the sensors (CMPR, PHYS, and CHEM), to provide control signals to the flow influencing device FID and the inflatable device INFL, and to administer medication to the victim. The administration of medication may be performed by means of a tube 1127 and a fitting, such as a Luer-fitting.

The external portion and the internal portion 1114 are connected by a catheter or catheter-like device 1110. The catheter 1110 groups the various connections between the internal portion 1114 and the external portion (control unit CU, medication administration tube 1127), which can be lumina, electrical conductors or mechanical links.

FIG. 11 also shows an automated cardio pulmonary resuscitation apparatus ACPR that is separate from the blood flow control device. Automated CPRs use techniques such as pneumatics to drive a compressing pad on to the chest of the patient. Another type of automated CPR is electrically powered and uses a large band around the patient's chest which contracts in rhythm in order to deliver chest compressions. Clinical studies have showed a marked improvement in coronary perfusion pressure and return of spontaneous circulation (ROSC). Since for the case of automated CPR the compression frequency is fixed and is controlled very accurately, the operation of the flow influencing element FID can easily be time synchronized. In this way a phase shift can be applied to the drive signal for the flow influencing element FID which could correct for the transition time between the flow state and the non-to-low-flow state (e.g. inflation time, deflation time, etc.).

The described and illustrated device is potentially useful both in-hospital and out-of-hospital. Some tendencies in current thinking state that CPR requires a more (minimally) invasive approach. Forward thinking suggests that with the advent of the guidelines 2010 separating lay and professional care more invasive applications will be sought. It aims to satisfy physical and information needs by professional caregivers involved in CPR. Potentially it may find application in other, low flow, conditions.

Other variations to the disclose embodiments can be understood and effected by those skilled in the art in practising the claimed invention from study of the drawings, the disclosure, and the appended claims. In the claims, the word “comprising” does not exclude other elements or steps, and the indefinite article “a” or “an” does not exclude a plurality. A single processor or other unit may perform functions of several items recited in the claims, and vice versa. The mere fact that certain measures are recited in mutually different dependent claims does not indicate that combination of these measures cannot be used to advantage. Any reference signs found in the claims should not be construed as limiting the scope.

1. Blood flow control device, comprising a flow influencing element arranged to be placed in the vena cava of a human during cardiopulmonary resuscitation and controllable between a non-to-low-flow state in which the flow influencing element substantially reduces a blood flow within the vena cava, and a flow state, in which the flow influencing element allows substantially unreduced blood flow, responsive to an existing or a predicted pressure difference between an upstream area and a downstream area of the flow influencing element,
wherein the flow influencing element comprises a functional valve, including two flaps adapted to move between a closed position in which the flow influencing element is in said non-to-low-flow state and an open position in which the flow influencing element is in said flow state.

2. Blood flow control device according to claim 1, further comprising:
a compression sensor, arranged to detect compressions related to an aspect of cardiopulmonary resuscitation, and
a control unit connected to the compression sensor and to the flow influencing element,
wherein the control unit, via the compression sensor, detects an intra thoracic pressure change or movement of the thoracic wall or compression, and sends a signal to the flow influencing element causing the flow influencing element to assume the non-to-low-flow state.

3. Blood flow control device according to claim 2, wherein the compression sensor is arranged to measure compression force and/or chest displacement and/or intra thoracic pressure change and/or intra vascular flow.

4. Blood flow control device according to claim 2, further comprising a catheter and wherein the compression sensor is placed in a tip of the catheter.

5. Blood flow control device according to claim 2, wherein the compression sensor is arranged to be positioned at the outside of the body of the human.

6. Blood flow control device according to claim 2, wherein the compression sensor is arranged to be positioned within the thoracic cavity.

7. Blood flow control device according to claim 1, further comprising at least one of a physiological sensor arranged to measure vital physiological parameters and a chemical sensor arranged to measure bio-chemistry parameters.

8. Blood flow control device according to claim 1, wherein the flow influencing element comprises an inflatable element or cusp shaped device

9. Blood flow control device according to claim 8, further comprising a pressure source and a pipe for connecting the pressure source with the inflatable element or the cusp shaped device for de/inflating the inflatable element or the cusp shaped device.

10. (canceled)

11. Blood flow control device according to claim 1, further comprising a catheter having a first lumen for the transmission of a drive signal to the flow influencing element for controlling the flow influencing element between the non-to-low-flow state and the flow state.

12. Blood flow control device according to claim 1, wherein the catheter further comprises at least a second lumen arranged to be used for the delivery of substances to a location in the vicinity of the flow influencing element.

13. Blood flow control device according to claim 1, wherein the flow influencing element functions in the manner of a check valve.

14. Blood flow control device according to claim 1, further comprising a control signal interface for receiving a control signal from an automated cardiopulmonary resuscitation apparatus, the control signal causing the flow influencing element to toggle between the non-to-low-flow state and the flow state in a synchronized manner with the automated cardiopulmonary resuscitation.

15. Blood flow control device according to claim 1, wherein the flow influencing element is arranged to be introduced into the vena cava by means of a femoral cannulation procedure.

16. Blood flow control device according to claim 1, wherein the flow influencing element remains in the flow state when ROSC is achieved or when chest compressions are paused or stopped.
